(19) United States
(12) Patent Application Publication
(10) Pub. No.: US 2017/0008902 A1
(43) Pub. Date: Jan. 12, 2017

(54) IMPROVED GLYCOL ACYLATION PROCESS WITH WATER-TOLERANT METAL TRIFLATES

(71) Applicant: ARCHER DANIELS MIDLAND COMPANY, Decatur, IL (US)

(72) Inventors: Kenneth Stensrud, Decatur, IL (US); Erik Hagberg, Decatur, IL (US); Erin Rockafellow, Decatur, IL (US); Stephen Howard, Sherman, IL (US)

(21) Appl. No.: 15/102,346

(22) PCT Filed: Dec. 11, 2014
(86) PCT No.: PCT/US14/69701

§ 371 Date: Jun. 7, 2016

Related U.S. Application Data
(60) Provisional application No. 61/918,172, filed on Dec. 19, 2013.

(51) Int. Cl. C07D 493/04; B01J 31/02

(52) U.S. Cl. CPC C07D 493/04; B01J 31/0232; B01J 2231/49; B01J 2531/004

(57) ABSTRACT

A method for acid-catalyzed acylation of an isohexide is described. The method can enable direct alcohol acylation with carboxylic acids. In particular, the method involves reacting an isohexide and an excess of carboxylic acid, in the presence of a water-tolerant Lewis acid catalyst. Water-tolerant Lewis acid catalysts can furnish relatively high diester yields (e.g., ≧55%-60%) at lower catalyst loads. This feature, among others, is highly desirable for cost savings, and can improve process economics.

### CLAIM BENEFIT OF PRIORITY

The present application claims benefit of priority of U.S. Provisional Patent Application No. 61/918,172, filed on Dec. 19, 2013, the contents of which are herein incorporated.

### FIELD OF INVENTION

The present disclosure relates to certain cyclic bi-functional materials that are useful as monomers in polymer synthesis, as well as intermediate chemical compounds. In particular, the present invention pertains to esters of 1,4:3,6-dianhydrohexitols and methods for their preparation.

### BACKGROUND

Traditionally, polymers and commodity chemicals have been prepared from petroleum-derived feedstock. As petroleum supplies have become increasingly costly and difficult to access, interest and research has increased to develop renewable or “green” alternative materials from biologically-derived sources for chemicals that will serve as commercially acceptable alternatives to conventional, petroleum-based or -derived counterparts, or for producing the same materials as produced from fossil, non-renewable sources.

One of the most abundant kinds of biologically-derived or renewable alternative feedstock for such materials is carbohydrates. Carbohydrates, however, are generally unsuited to current high temperature industrial processes. Compared to petroleum-based, hydrophobic aliphatic or aromatic feedstocks with a low degree of functionalization, carbohydrates such as polysaccharides are complex, over-functionalized hydrophilic materials. As a consequence, researchers have sought to produce biologically-based chemicals that can be derived from carbohydrates, but which are less highly functionalized, including more stable bi-functional compounds, such as 2,5-furandicarboxylic acid (FDCA), levulinic acid, and 1,4:3,6-dianhydrohexitols.

1,4:3,6-Dianhydrohexitols (also referred to herein as isohexides) are derived from renewable resources from cereal-based polysaccharides. Isohexides embody a class of bicyclic furanodiols that derive from the corresponding reduced sugar alcohols (D-sorbitol, D-mannitol, and D-iditol respectively). Depending on the chirality, three isomers of the isohexides exist, namely: A) isosorbide, B) isomannide, and C) isoidide, respectively; the structures of which are illustrated in Scheme A.

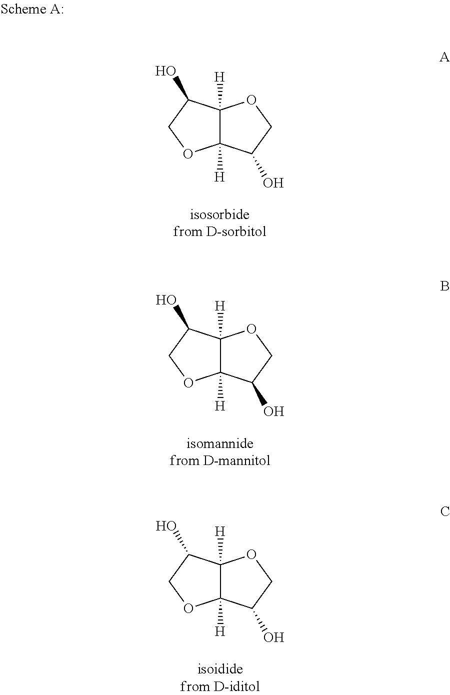 [SMILES: O[C@@H]1CO[C@H]2[C@@H]1OC[C@@H]2O ; O[C@@H]1CO[C@H]2[C@@H]1OC[C@H]2O ; O[C@H]1CO[C@H]2[C@@H]1OC[C@@H]2O]

These molecular entities have received considerable interest and are recognized as valuable, organic chemical scaffolds for a variety of reasons. Some beneficial attributes include relative facility of their preparation and purification, the inherent economy of the parent feedstocks used, owing not only to their renewable biomass origins, which affords great potential as surrogates for non-renewable petrochemicals, but perhaps most significantly the intrinsic chiral bi-functionalities that permit a virtually limitless expansion of derivatives to be designed and synthesized.

The isohexides are composed of two cis-fused tetrahydrofuran rings, nearly planar and V-shaped with a 120° angle between rings. The hydroxyl groups are situated at carbons 2 and 5 and positioned on either inside or outside the V-shaped molecule. They are designated, respectively, as endo or exo. Isoidide has two exo hydroxyl groups, while the hydroxyl groups are both endo in isomannide, and one exo and one endo hydroxyl group in isosorbide. The presence of the exo substituents increases the stability of the cycle to which it is attached. Also exo and endo groups exhibit different reactivities since they are more or less accessible depending on the steric requirements of the derivatizing reaction.

As interest in chemicals derived from natural resources is increases, potential industrial applications have generated interest in the production and use of isohexides. For instance, in the field of polymeric materials, the industrial applications have included use of these diols to synthesize or modify polycondensates. Their attractive features as monomers are linked to their rigidity, chirality, non-toxicity, and the fact that they are a bio-renewable feedstock. For these reasons, the synthesis of high glass transition temperature polymers with good thermo-mechanical resistance and/or with special optical properties is possible. Also the innocuous character of the molecules opens the possibility of applications in packaging or medical devices. For instance, production of isosorbide at the industrial scale with a purity satisfying the requirements for polymer synthesis suggests that isosorbide can soon emerge in industrial polymer applications. (See e.g., F. Fenouillot et al., “Polymers From Renewable 1,4:3,6-Dianhydrohexitols (Isosorbide, Isommanide and Isoidide): A Review,” PROGRESS IN POLYMER SCIENCE, vol. 35, pp. 578-622 (2010); or X. Feng et al., “Sugar-based Chemicals for Environmentally sustainable Applications,” CONTEMPORARY SCIENCE OF POLYMERIC MATERIALS, Am. Chem. Society, December 2010; or isosorbide-based plasticizers, e.g., U.S. Pat. No. 6,395,810, contents of each are incorporated herein by reference.)

Isohexide esters are being vigorously pursued as renewable surrogates to petro-based incumbents in the realm of plasticizers, dispersants, lubricants, flavoring agents, solvents, etc. The established commercial synthesis of esters entails direct alcohol acylation with carboxylic acids catalyzed by a Bronsted or Lewis acid, this protocol commonly specified as the Fischer-Speier esterification. Typically, strong inorganic acids such as H₂SO₄, H₃PO₄, and HCl are employed as the catalyst. These strong acids are readily obtained, inexpensive materials but are difficult to regenerate. Additionally, these acids can react in an undesired manner by the addition of their anionic moiety forming biproducts such as sulfate esters.

In order to avoid the regeneration and attendant disposal problems, solid resin catalysts have been tried. Unfortunately, in the presence of water and at the temperatures required for carrying out the dehydration, very few solid acids can demonstrate the activity and stability needed to begin to contemplate a commercially viable process. Furthermore, traditionally employed solid acids are not hydrolytically stable and even trace amounts of water can negatively impact the catalytic activity.

In order to achieve optimum target yields, catalyst loadings typically span 1 to 10 wt. % per alcohol functionality. Improved catalyst proficiency, i.e., preserving high ester yields with reduced catalyst loadings, is highly desirable from the standpoint of process economics.

### SUMMARY OF INVENTION

The present disclosure describes, in part, a method for synthesizing esters from isohexide compounds. Generally, the method encompasses performing a Fischer esterification with an isohexide and a carboxylic acid in the presence of a water-tolerant Lewis acid catalyst at a temperature up to about 250° C. for a period of less than about 24 hours. The method uses reduced catalyst loads of the Lewis acid, as it does not appreciably lose its catalytic efficacy in the presence of water. The isohexide is converted at a rate of ≧50 wt. %, and produces a diester yield of at least 10 wt. % relative to the isohexide.

Particular water-tolerant Lewis acids can manifest high catalytic activity in acylating isohexides, such as with 2-ethylhexanoic acid, at markedly diminished catalyst loadings vis a vis results from the currently favored incumbent, sulfuric acid. The amount of Lewis acid catalyst load can range from being very low (e.g., 0.0001 wt. %) up to about 10 wt. % relative to isohexide content. Typically, the amount of catalyst loading is less than about 2.0 wt. % or about 1.0 wt. %; more typically it can be up to about 0.5 wt. % or 0.8 wt. %. The isohexide is converted to a corresponding ester product at a relatively high rate of conversion (e.g., ≧50 wt. %, 55 wt. %, or 60 wt. %), and the ester product mixture contains isohexide diesters, at a relatively high yield (e.g., ≧60 wt. %).

In another aspect, the present disclosure pertains to water-tolerant Lewis acid catalysts. In particular embodiments, the water-tolerant catalysts can be one or more metallic triflates (e.g., aluminum, tin, indium, hafnium, gallium, scandium, or bismuth triflates). The Lewis acid catalyst can be either homogenous or heterogenous catalyst.

In yet another aspect, the present disclosure describes a method of preparing an ester of an isohexide directly from a sugar alcohol in a single reaction vessel. The method involves providing a sugar alcohol in a single reaction vessel with an excess of carboxylic acid in the presence of a water-tolerant Lewis acid catalyst; melting the sugar alcohol to form a biphasic system, in which the molten sugar alcohol and Lewis acid catalyst are in a lower phase and the carboxylic acid is in an upper phase; and dehydrating the sugar alcohol in its own phase to form an isohexide. Allow the isohexide along with said Lewis acid catalyst to migrate into the carboxylic acid phase, in which the isohexide contacts with the carboxylic acid at a reaction temperature and for a time sufficient to produce a mixture of corresponding ester derivatives of the isohexide.

Additional features and advantages of the present purification process will be disclosed in the following detailed description. It is understood that both the foregoing summary and the following detailed description and examples are merely representative of the invention, and are intended to provide an overview for understanding the invention as claimed.

### BRIEF DESCRIPTION OF FIGURES

FIG. 1 is a graph that shows the relative rates of conversion of isosorbide over time per catalyst loading at 0.01 wt. %, for metal triflates (bismuth, gallium and scandium) as compared to sulfuric acid.

FIG. 2 is a graph that shows the relative rates of conversion of isosorbide over time per catalyst loading at 0.001 wt. %, of the catalyst species in FIG. 1.

FIG. 3 is a graph that shows the resultant yields of isosorbide diesters from acylation reactions performed using catalyst loadings at 0.01 wt. % for the respective catalyst species.

FIG. 4 is a graph that shows the resultant yields of isosorbide diesters performed using catalyst loadings at 0.001 wt. % for the respective catalyst species.

FIG. 5A is a graph that shows compares the relative conversion rate of isosorbide over time using four species of trilfates (hafnium, gallium, scandium, and bismuth) as compared to sulfuric acid.

FIG. 5B is a graph that shows the resultant yields of isosorbide diesters from acylation reactions performed using catalyst loadings at 0.01 wt. % for the respective catalyst species.

### DETAILED DESCRIPTION OF INVENTION

### I. Description

As biomass derived compounds that afford great potential as surrogates for non-renewable petrochemicals, 1,4:3,6-dianhydrohexitols are a class of bicyclic furanodiols that are valued as renewable molecular entities. (For sake of convenience, 1,4:3,6-dianhydrohexitols will be referred to as “isohexides” in the Description hereinafter.) As referred to above, the isohexides are good chemical platforms that have recently received interest because of their intrinsic chiral bi-functionalities, which can permit a significant expansion of both existing and new derivative compounds that can be synthesized.

Isohexide starting materials can be obtained by known methods of making respectively isosorbide, isomannide, or isoidide. Isosorbide and isomannide can be derived from the dehydration of the corresponding sugar alcohols, D-sorbitol and D mannitol. As a commercial product, isosorbide is also available easily from a manufacturer. The third isomer, isoidide, can be produced from L-idose, which rarely exists in nature and cannot be extracted from vegetal biomass. For this reason, researchers have been actively exploring different synthesis methodologies for isoidide. For example, the isoidide starting material can be prepared by epimerization from isosorbide. In L. W. Wright, J. D. Brandner, J. Org. Chem., 1964, 29 (10), pp. 2979-2982, epimerization is induced by means of Ni catalysis, using nickel supported on diatomaceous earth. The reaction is conducted under relatively severe conditions, such as a temperature of 220° C. to 240° C. at a pressure of 150 atmosphere. The reaction reaches a steady state after about two hours, with an equilibrium mixture containing isoidide (57-60%), isosorbide (30-36%) and isomannide (5-7-8%). Comparable results were obtained when starting from isoidide or isomannide. Increasing the pH to 10-11 was found to have an accelerating effect, as well as increasing the temperature and nickel catalyst concentration. A similar disclosure can be found in U.S. Pat. No. 3,023,223, which proposes to isomerize isosorbide or isomannide. More recently, P. Fuertes proposed a method for obtaining L-iditol (precursor for isoidide), by chromatographic fractionation of mixtures of L-iditol and L-sorbose (U.S. Patent Publication No. 2006/0096588; U.S. Pat. No. 7,674,381 B2). L-iditol is prepared starting from sorbitol. In a first step sorbitol is converted by fermentation into L-sorbose, which is subsequently hydrogenated into a mixture of D-sorbitol and L-iditol. This mixture is then converted into a mixture of L-iditol and L-sorbose. After separation from the L-sorbose, the L-iditol can be converted into isoidide. Thus, sorbitol is converted into isoidide in a four-step reaction, in a yield of about 50%. (The contents of the cited references are incorporated herein by reference.)

### A. Preparation of Isohexide Diesters

The Fischer-Speier esterification typifies the standard protocol for industrial preparation of esters in operations that employ acid catalysts in amounts that typically exceed about 10 wt. %. The present disclosure describes a transformation that uses water-tolerant Lewis acid catalysts at lower catalysts loads, which can enable a facile process for direct alcohol acylation with carboxylic acids. Water-tolerant Lewis acids are receiving much attention in effectuating a multitude of chemical transformations, and are reviewed thoroughly, in Chem Rev, 2002, 3641-3666, the contents of which are incorporated herein by reference. The present discovery that these catalysts can furnish relatively high diester yields (e.g., ≧55%-60%) at lower loads is highly desirable, and can ameliorate process economics.

In contrast to currently practiced commercial esterification protocols, which typically involve at least 1 wt. % catalyst loadings, the esterification method according to the present invention, may use catalysts in amounts of two or three orders of magnitude less to achieve congruent yields of diesters, and hence are suitable in terms of moderating cost while concurrently augmenting the overall process efficiency. The metal triflate catalyst can be present in an amount of at least 0.0001 wt. % relative to the amount of isohexide.

Traditionally, Lewis acids favor conditions in which virtually no water moisture is present, as they can quickly hydrolyze and lose their catalytic function even in with minor or trace amounts of water. As used herein, the term “water-tolerant” refers to a characteristic of a metal ion of a particular catalyst to resist being hydrolyzed by water to a high degree. Metal triflates possess this remarkable trait, (e.g., see, J. Am. Chem. Soc. 1998, 120, 8287-8288, the content of which is incorporated herein by reference). Water-tolerant Lewis acids, for example, may include one or more of metal triflates (e.g., triflates of Al, Sn (II), In (III), Fe (II), Cu (II), Zn (II), Bi (III), Ga (III), Sc (III), Y (III), La (III)), Hf (IV) triflates). (Lewis acid activity in descending order: Hf>Ga>Sc>Bi>In>Al>Sn.) Other metal triflate species may include: Lanthanide rare-earth metal triflates (cerium, praseodymium, neodymium, samarium, europium, gadolinium, terbium, dysprodium, holmium, erbium, ytterbium, lutetium), and/or transitional metal triflates (hafnium, mercury, nickel, zinc, thallium, tin, indium), or a combination of any of the foregoing metal triflates.

In the present method, the isohexide can be at least one or more of the following: isosorbide, isomannide, and isoidide. The carboxylic acid can be at least an alkanoic acid, alkenoic acid, alkynoic and aromatic acid, having C₂-C₂₆. Although the following description and examples use isosorbide as an isohexide species for purpose of illustration, the present invention is not so limited but is also applicable equally to other isohexides: isomannide and isoidide.

Scheme 1 delineates the synthetic methodology for isosorbide esterification with these catalysts.

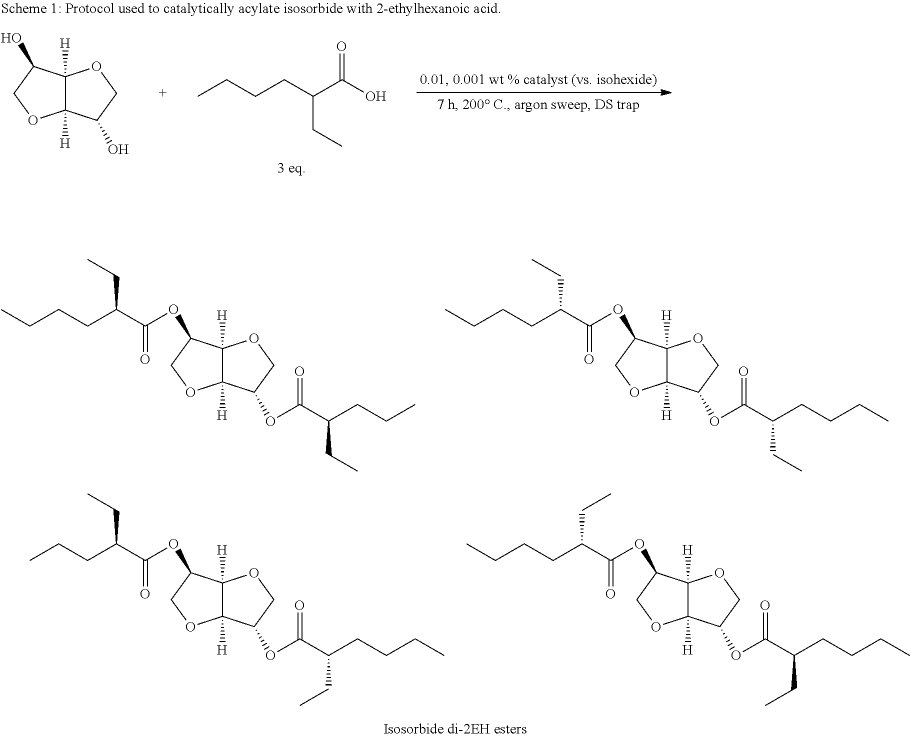

In certain embodiments, the water-tolerant Lewis acid catalyst is a metal triflate, and the acylating agent is a carboxylic acid (e.g., 2-ethylhexanoic acid). In embodiments, the method can use catalyst in amounts as low as about 0.01 wt. %, with ensuing full conversion of isohexides (e.g., isosorbide) to corresponding diesters, in >80% yields. Alternatively, the method may use catalysts in amounts as low as about 0.001 wt. %, with isosorbide conversions of >80%, and diester yields >10%.

An advantage of the present Lewis acid catalysts (metal triflates), is that these catalysts can be recovered and reused. The diester product is not water soluble; therefore, the catalyst can be removed with a water wash and recovered by removal of the water similar to the process described in U.S. Patent Application Publication No. 2013/0274389 A1, the content of which is incorporated herein by reference.

In other examples, the amounts of catalyst loadings are about 0.01 wt % and 0.001 wt. %, manifesting a greater degree of isosorbide conversions and diester yields at the former catalyst loading levels. The esterification is performed at a temperature in a range from about 150° C. or 160° C. to about 240° C. or 250° C. Typically, the reaction temperature interval is 170° C. or 175° C. to about 205° C. or 220° C. In other embodiments, when the amount catalyst is at least 0.005 wt %, the diesters preponderate in the product mixture. In other embodiments, when the catalyst is present in an amount from about 0.001 to 0.005 wt. %, the product mixture contains about a 1:1 ratio of monoesters and diesters. In still other embodiments, when the amount of catalyst is present in an amount <0.001 wt. %, the product mixture contains predominantly monoesters and unreacted isosohexide.

The reactions according to the present methods can be performed from about 1 to about 24 hours. Typically, a reaction is conducted between about 2-12 hours, more typically within about 8 or 10 hours (e.g., 2-5 or 7 hours). With optimization in certain embodiments at reaction times of about 300 minutes or more, one can achieve isohexide conversions of about 60% or 70% to about 98%.

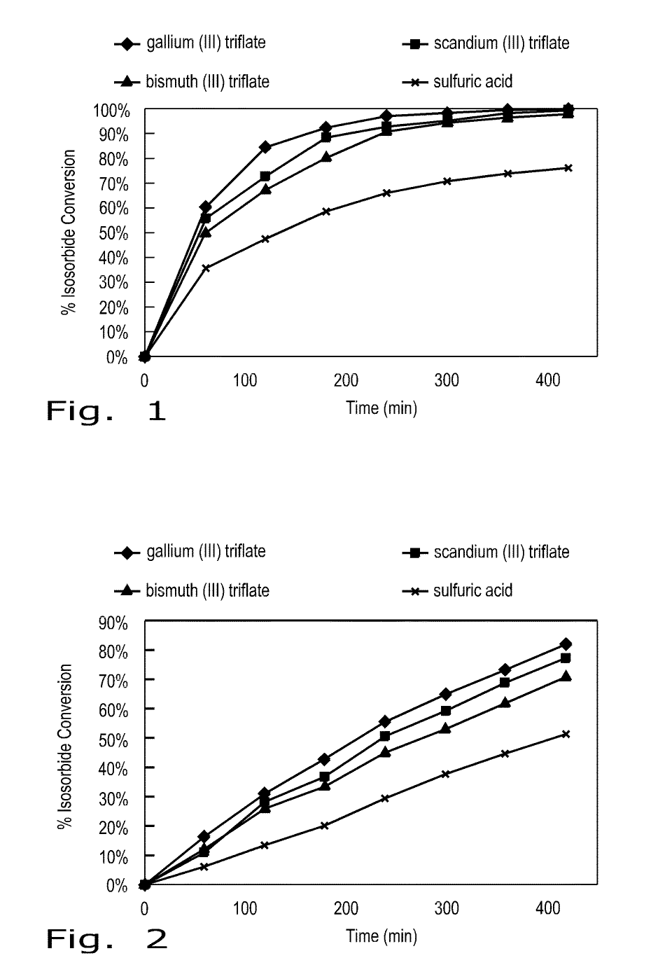

FIG. 1, presents the comparative isosorbide conversions over time as a function of catalyst type at loadings of 0.01 wt. %. The metal triflates display quantitative conversions (i.e., ˜100%) of isosorbide. Specifically, hafnium and gallium triflates manifested the highest conversion in the least amount of time, 300 minutes, followed by scandium triflate, 360 minutes, then bismuth triflate, 420 minutes. While, for sake of comparison a Bronsted acid, sulfuric acid, produced only ˜70% at 7 hours.

In FIG. 2, an analogous comparison is made at 0.001 wt. % catalyst loadings. The reaction rates for each catalyst species slowed but the overall patterns are maintained from that shown in FIG. 1. Correspondingly, the metal triflates tendered a higher isosorbide conversion than sulfuric acid. Specifically, gallium triflate afforded the highest conversion, 82%, followed by scandium triflate, 78%, and bismuth triflate, 70%, while sulfuric acid provided only a 50% conversion. The change in rate suggests that one can adjust the amount of catalyst loading to control the respective amounts of monoester and diester produced.

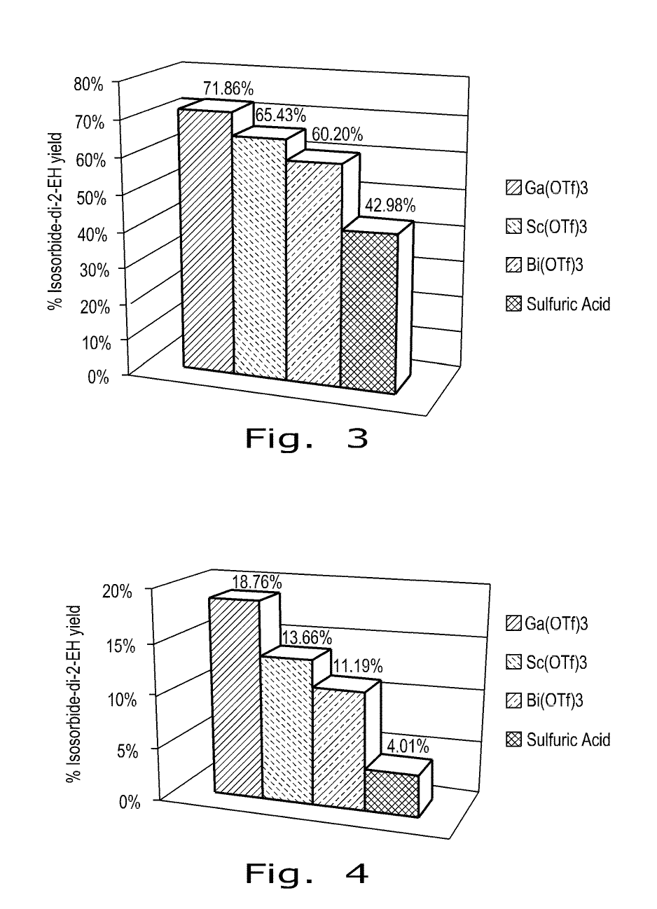

FIG. 3 displays the resulting yields of isosorbide diesters as compared per catalyst loadings at 0.01 wt. %. Analogous to isosorbide conversion, the metal triflates performed superiorly in affording isosorbide diesters. Specifically, gallium exhibited the highest potency, furnishing a 72% yield, followed by scandium, 65%, then bismuth 60%. The incumbent, sulfuric acid, was the most static, furnishing a 43% diester yield. Comparisons were also distinguished at 0.001 wt. %, summarized in FIG. 4. Again, the metal triflates expressly manifested the highest activity vis a vis sulfuric acid. In particular, gallium are the most cogent, affording about 19% diester yields, respectively, followed by scandium, 14%, and bismuth 11%. Sulfuric acid evinced the least catalytic activity, engendering only a 4% diester yield.

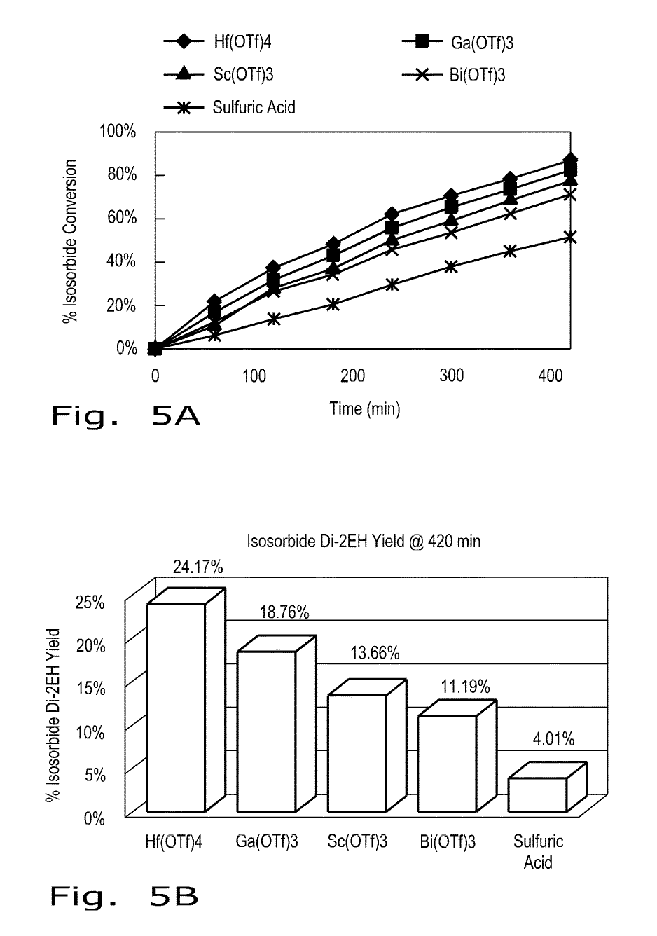

In another embodiment, one can also employ the triflate of hafnium, which has a valence of 4+. As depicted in accompanying FIG. 5A, this species exhibits fast reactivity and good selectivity for isosorbide diester yields, better than the other species having 3+ valence (i.e., Ga, Sc, Bi), even at relatively low levels of catalysts-loading (0.001 wt. %). After about 400 minutes of reaction, the triflates are able to manifest between about 70% to about 85% or 86% conversion of the isosorbide, in comparison to about 50% using sulfuric acid, the conventional catalyst. FIG. 5B shows the respective yield of isosorbide diester achieved using the different catalysts species after reacting for about 420 minutes. A reaction using the hafnium triflate (24.17%) produced about 16.67% (⅙) more diester than a reaction using the gallium triflate (18.76%), which in turn was about a sixth greater than the yield from the scandium triflate (13.66%). All of the triflate species exhibited greater yield over sulfuric acid (4.01%). Table 1 summaries the respective conversion rates and yield of isosorbide diesters for selective triflate species in reaction over time of 0-420 minutes.

TABLE 1

| | | | | | Sulfuric |
|---|---|---|---|---|---|
| Time (Min.) | Hf(OTf)₄ | Ga(OTf)₃ | Sc(OTf)₃ | Bi(OTf)₃ | acid |
| 0 | 0.00% | 0.00% | 0.00% | 0.00% | 0.00% |
| 60 | 21.50% | 16.63% | 11.20% | 12.30% | 5.94% |
| 120 | 36.82% | 31.07% | 28.35% | 26.51% | 13.30% |
| 180 | 48.01% | 42.96% | 36.92% | 34.26% | 20.10% |
| 240 | 61.73% | 55.55% | 50.50% | 45.63% | 29.40% |
| 300 | 70.05% | 64.92% | 59.38% | 53.26% | 37.75% |
| 360 | 77.92% | 73.41% | 68.95% | 62.04% | 44.83% |
| 420 | 86.36% | 82.02% | 77.58% | 70.98% | 51.32% |
| Diester yield | 24.17% | 18.76% | 13.66% | 11.19% | 4.01% |

Another advantageous feature of the present methods is the ability to perform the esterification from a sugar alcohol directly, as well as from an isohexide. According to an embodiment, the conversion of a sugar alcohol to its isohexide cyclic derivative and subsequent etherification can be performed all in a single reaction vessel (i.e., “one pot”). One can start with solid metal triflate and a solid sugar alcohol, such as sorbitol instead of isosorbide, with a liquid carboxylic acid. At the outset, molten sorbitol and carboxylic acid form a biphasic system, with the carboxylic acid in an upper phase layer and denser sorbitol in a lower phase layer. The Lewis acid catalyst is in the sorbitol layer due to dipole-electrostatic attractions. Mediated by the Lewis acid catalyst, sorbitol then dehydrates in its own phase to form isosorbide, which diffuses, along with the catalyst into the carboxylic acid layer. Immured in the carboxylic acid layer, isosorbide then undergoes catalytic acylation.

For example, an amount of sorbitol is added to a three neck round bottomed flask equipped with a PTFE coated magnetic stir bar. To the sorbitol is added 0.1 mol. % (relative to the concentration of sorbitol) of solid metal triflate catalyst, followed by a volume of 2-ethylhexanoic acid that corresponds to three molar equivalents. To the rightmost neck is affixed a ground glass adapted argon inlet, the center neck a thermowell adapter, and the leftmost neck a jacketed Dean-Stark trap filled with 2-ethylhexanoic acid and capped with a 14″ needle-permeated rubber septum (argon outlet). While vigorously stirring, the sorbitol suspension mixture is heated to about 175° C. At about 100° C. point, the sorbitol is observed to melt, the result of which is a clear phase separation. The high polarity of molten sorbitol is believed to be the electrostatically preferable medium for the triflate salt. This is corroborated by the fact that no suspended solids were manifest in an upper carboxylic acid layer. At approximately 150° C., a profusion of water began to assimilate in the glass tubing of the DS trap while the biphasic feature is maintained, this shows the two-fold dehydrative cyclization of sorbitol to isosorbide. In the example, the sugar alcohol (sorbitol) is complete conversed to isosorobide, and the biphasic quality of the mixture transforms into a single phase. This consistent with another aspect of the present invention where the solubility of isosorbide in 2-ethylhexanoic acid at 175° C. is demonstrated. The matrix darkened to a dull brown over the remaining 2 hours of the reaction, at which time aliquots were removed and analyzed by GC.

Examples of “one pot” esterification of sorbitol to isosorbide mono and di-2-ethylhexanoates using different metal triflate catalysts are summarized in Table 2. In the table, phosphonic acid (H₃PO₃) is a comparative example. The percent product accountability refers to the fractional amount of a reaction product mixture that is a knowable component including unreacted starting isohexides, and mono- and/or diesters, less any unspecified byproducts.

TABLE 2
One pot sorbitol conversion to isosorbide mono and di-2-ethylhexanoate
GC-Silanation Analysis

| | | | Catalyst | | | | isosorbide | isosorbide | % product |
|---|---|---|---|---|---|---|---|---|---|
| | | | load | time | temp | isosorbide | mono 2EH | di 2EH | account- |
| Run | *solvent | catalyst | (mol. %) | (h) | (° C.) | (wt. %) | (wt. %) | (wt. %) | ability |
| 1 | xylenes | Bi(OTf)₃ | 0.1 | 4 | 170 | 1.50 | 3.54 | 0.00 | 93.29 |
| 2 | 2EH | Bi(OTf)₃ | 0.1 | 3 | 170 | 2.52 | 18.41 | 21.21 | 91.24 |
| 3 | 2EH | H₃PO₃ | 10 | 3 | 170 | 2.09 | 13.22 | 8.94 | 61.46 |
| 4 | 2EH | In(OTf)₃ | 0.1 | 3 | 170 | 1.80 | 17.10 | 25.07 | 92.16 |
| 5 | 2EH | Al(OTf)₃ | 0.1 | 3 | 170 | 1.86 | 17.44 | 25.38 | 91.48 |
| 6 | 2EH | **AgOTf | 0.1 | 3 | 170 | 5.71 | 8.90 | 0.92 | 89.04 |
| 7 | 2EH | **La(OTf)₃ | 0.1 | 3 | 170 | 1.90 | 3.03 | 0.41 | 96.44 |
| 8 | 2EH | **Fe(OTf)₂ | 0.1 | 3 | 170 | 0.20 | 0.71 | 0.00 | 97.32 |
| 9 | 2EH | Ga(OTf)₃ | 0.1 | 3 | 170 | 1.04 | 15.14 | 34.02 | 89.60 |
| 10 | 2EH | **Zn(OTf)₂ | 0.1 | 3 | 170 | 2.15 | 8.76 | 1.23 | 93.89 |
| 11 | 2EH | Sc(OTf)₃ | 0.1 | 3 | 170 | 5.25 | 20.62 | 11.28 | 92.13 |
| 12 | 2EH | Sn(OTf)₂ | 0.1 | 3 | 170 | 4.32 | 17.17 | 23.04 | 93.65 |
| 13 | 2EH | Hf(OTf)₄ | 0.1 | 3 | 170 | 1.33 | 13.22 | 36.95 | 90.74 |

*4 mol. equivalents 2EH per sorbitol
**Biphasic product mixture

While we have described in the foregoing the present inventive concept in terms of isohexides, it is understood that the present disclosure is not necessarily limited to use only for those particular substrates. It is envisioned that, for instance, the processing of a variety of carbohydrate-derived cyclic ethers and/or other isolable platforms from sorbitol hydrogenation/hydrogenolysis can benefit from these water-tolerant Lewis acid catalysts, which generally maintain their catalytic efficacy in the presence of water, or under hydrolysis conditions. Some other substrates may include other carbohydrate-derive cyclic ethers, for example: sorbitan; or other polyols: 1,2,5,6-hexanetetrol, 1,2,5-hexanetriol, 1,6-hexanediol.

The present invention has been described in general and in detail by way of examples. Persons of skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein.

1) A method for acid-catalyzed acylation of an isohexide, comprising contacting an isohexide with an excess of carboxylic acid in the presence of a water-tolerant Lewis acid catalyst at a reaction temperature and for a time sufficient to produce a mixture of corresponding ester derivatives of isohexide, wherein said isohexide is transformed to said ester derivatives at a conversion rate of ≧50 wt. %.

2) (canceled)

3) The method according to claim 1, wherein said reaction temperature ranges from about 150° C. to about 250° C.

4) The method according to claim 3, wherein said reaction temperature ranges from 170° C. to 220° C.

5) The method according to claim 1, wherein said reaction time is less than about 24 hours.

6) The method according to claim 5, wherein said reaction time is between about 1-12 hours.

7) (canceled)

8) The method according to claim 1, wherein said isohexide conversion rate is from about 55% to 100%.

9) The method according to claim 8, wherein said isohexide conversion rate is about 60% to about 98%.

10) The method according to claim 1, wherein said ester product mixture contains isohexide diesters at a yield of at least ≧5 wt. % relative to the isohexide content.

11) The method according to claim 10, wherein said yield of isohexide diester ranges from about 50% to about 85% relative to the isohexide content.

12) The method according to claim 11, wherein said yield of diester is about 70% to about 75% relative to the isohexide content.

13) The method according to claim 1, wherein said isohexide is at least one of isosorbide, isomannide, and isoidide.

14) The method according to claim 1, wherein said carboxylic acid is selected from the group consisting of an alkanoic, alkenoic, alkyonoic, and aromatic acid, having a carbon chain length ranging from C₂-C₂₆.

15) The method according to claim 1, wherein said carboxylic acid is present in about 2-fold to about 10-fold molar excess relative to the isohexide.

16) The method according to claim 15, wherein said carboxylic acid is present in about 3-fold molar excess relative to the isohexide.

17) The method according to claim 1, wherein said water-tolerant Lewis acid catalyst is either a homogenous or a heterogenous catalyst.

18) The method according to claim 1, wherein said Lewis acid catalyst is a water-tolerant metal triflate, selected the group consisting of: lanthanum, cerium, praseodymium, neodymium, samarium, europium, gadolinium, terbium, dysprodium, holmium, erbium, ytterbium, lutetium, gallium, scandium, bismuth, hafnium, mercury iron, nickel, copper, zinc, thallium, tin, and indium triflate, or a combination thereof.

19) The method according to claim 18, wherein said the metal triflate is at least one of: hafnium, gallium, scandium, and bismuth triflate.

20) The method according to claim 1, wherein said metal triflate is present in an amount of catalyst loading that ranges from about 0.0001 mol. % to about 10 mol. % of the isohexide.

21) The method according to claim 20, wherein said metal triflate is present in an amount of catalyst loading that ranges from about 0.001 mol. % to about 0.01 mol. % relative to the isohexide content.

22) The method according to claim 1, wherein said acid-catalyzed acylation is performed in a single reaction vessel as a biphasic system.

23) The method according to claim 22, wherein said biphasic system is composed of a denser sugar alcohol in a lower phase layer and a carboxylic acid in an upper phase layer in said single reaction vessel.

24) The method according to claim 23, wherein said sugar alcohol is transformed into said isohexide and migrates into a single phase with said carboxylic acid.

25) A method of preparing an ester of an isohexide comprising: providing a sugar alcohol in a single reaction vessel with an excess of carboxylic acid in the presence of a water-tolerant Lewis acid catalyst; melting said sugar alcohol to form a biphasic system, in which said molten sugar alcohol and Lewis acid catalyst are in a lower phase and said carboxylic acid is in an upper phase; dehydrating said sugar alcohol in its own phase to form an isohexide; migrating said isohexide along with said Lewis acid catalyst into said carboxylic acid phase, in which said isohexide contacts with said carboxylic acid at a reaction temperature and for a time sufficient to produce a mixture of corresponding ester derivatives of said isohexide.
